(19) United States
(12) Patent Application Publication
(10) Pub. No.: US 2009/0068706 A1
(43) Pub. Date: Mar. 12, 2009

(54) Production of Highly Isotopically Labelled, Secondary, Microbial Metabolic Products, and Corresponding Metabolic Products

(76) Inventors: Martin Freudenschuss, Ybbs (AT); Georg Haeubl, Vienna (AT); Rudolf Krska, Tulln (AT); Guenther Jaunecker, Tulln (AT); Eva Binder, Unterwart (AT)

Correspondence Address: JACOBSON HOLMAN PLLC, 400 SEVENTH STREET N.W., SUITE 600, WASHINGTON, DC, 20004, US

(21) Appl. No.: 11/887,149

(22) PCT Filed: Mar. 31, 2006
(86) PCT No.: PCT/AT2006/000129

§ 371 Date: Sep. 26, 2007

(30) Foreign Application Priority Data
Apr. 5, 2005 (AT) A 574/2005

(51) Int. Cl. C12P 21/00

(52) U.S. Cl. 435/71.3

(57) ABSTRACT

The invention relates to a method for producing isotopically labelled secondary metabolic products of fungi or bacteria in a liquid synthetic culture medium. According to said method, the synthesis is carried out by immobilizing the fungi or bacteria on an inert carrier, adding a liquid synthetic culture medium in which essentially all of the carbon atoms, nitrogen atoms and/or sulphur atoms are replaced by stable isotopes.

The present invention relates to a method for producing isotopically labelled secondary metabolic products of fungi or bacteria in a liquid synthetic culture medium as well as isotopically labelled secondary metabolic products of fungi or bacteria.

Today, isotopically labelled substances are of increasing importance, in particular in the technology of liquid chromatography with mass spectrometric detection (LCMS), which enables efficient spectrometric analyses at high product throughputs. This technology can be used for a great variety of potential analytes without imposing any limitations as to the molecular mass, yet with possible problems occurring in the detection of the individual substances to the effect that both the disintegration spectra and the individual molecule peaks have to be assigned accordingly. In order to ensure a reliable LCMS application and method, the use of so-called internal standard substances has become increasingly important. Internal standards are substances strongly resembling the target analytes proper, i.e., in particular, possibly having identical molecular structures yet at different molecular weights. Isotopically labelled molecules of the target analyte, i.e. molecules in which one or several atoms in the molecule are replaced by their isotopes, therefore, have turned out to be ideal internal standards. At present, such substances are produced by organic syntheses, for instance, by substituting hydrogens or carbons with the corresponding, heavier isotopes.

In this context, it has, however, been shown in LCMS analyse that it is desirable that the isotopically labelled substances used as internal standards have molecular mass differences of at least 3 in order to enable the distinct separation from the target analytes, and that, if possible, substances comprising as few isotopomers as possible are to be used.

A way of producing isotopically labelled plant or microbial metabolites is via the biosynthetic path of the respective plants and/or microbes. In doing so, culture media are supplemented with radioactively labelled nutrients and the culture medium components are to a certain percentage integrated in the anabolic and metabolic cycles of the microbial or plant cultures such that isotopes will be incorporated in the metabolic products. That method involves the drawback that only incomplete labelling is feasible by this method and that a mixture of different isotopomers is normally formed, what makes such isotopically labelled substances, or isotopically labelled plant or microbial metabolites, hardly suitable for use as internal standards, since with the use of such substances not one standard but a broad spectrum of isotopomers would be applied, which would, in turn, render the selective detection of target substances in LCMS spectrometries possible not at all or only with great difficulty.

The present invention aims to provide a method for producing isotopically labelled secondary metabolic products of fungi or bacteria, in which all or almost all of the carbon atoms, nitrogen atoms or sulphur atoms contained the starting product are replaced by stable isotopes, thus providing a single, isotopically labelled end product to be readily and reliably detectable in spectrometric processes, in particular LCMS. The invention further aims to produce a metabolic product which can be safely and reliable used as an internal standard in spectrometric analytical processes, in particular LCMS.

To solve these objects, the method according to the present invention is conducted in a manner that the synthesis is carried out by immobilizing the fungi or bacteria on an inert carrier while adding a liquid synthetic culture medium in which substantially all of the carbon atoms, nitrogen atoms and/or sulphur atoms have been replaced by stable isotopes. By realizing the synthesis by immobilizing the fungi or bacteria on an inert carrier while adding a liquid synthetic culture medium in which substantially all of the carbon atoms, nitrogen atoms and/or sulphur atoms have been replaced by stable isotopes, it has become feasible to produce an isotopically labelled metabolic product of the fungi and bacteria, in which all of the atoms, or at least 95% of the atoms, to be obtained from the culture medium by growing, i.e. carbon, nitrogen or sulphur atoms, have been replaced by the more stable isotopes of the isotopically labelled nutrients contained in the culture medium so as to allow for the recovery of a selective, isotopically labelled product rather than a mixture of different homologs having varying numbers of isotope atoms, as was frequently described in the prior art. It is, thus, feasible by this production method to obtain an isotopically labelled secondary metabolic product which can be selectively used and which can be clearly and unambiguously detected in any analysis, even in metabolic studies.

According to a further development, the method is conducted in a manner that sugars or sugar alcohols, in particular D-[U-¹³C₆]-glucose, ¹³C-sucrose, ¹³C-gycerol and/or ¹³C-acetate are used as carbon sources in the liquid synthetic culture medium, ¹⁵N-amino acids, -nitrates, -ammonium compounds or -urea are used as nitrogen sources, ³³S- or ³⁴S-sulphates, -sulphides or -amino acids are used as sulphur sources. When growing metabolic products of fungi or bacteria, the fungus or the bacterium, respectively, due to completely labelled carbon, nitrogen and/or sulphur sources being contained in the liquid synthetic culture medium, will be forced to incorporate into the metabolic product the respectively labelled isotope so as to ensure that the secondary metabolic products of the fungi or bacteria will be labelled with the respective isotopes, or replaced by the respected isotopes, to a high degree, if not completely.

In order to improve the yield of isotopically labelled secondary metabolic products of fungi or bacteria, the method according to a further development is conducted in a manner that the liquid synthetic culture medium additionally contains a mixture selected from inorganic salts or acids and bases having the ions Na⁺, K⁺, Ca⁺⁺, Mg⁺⁺, Fe⁺⁺⁺, Zn⁺⁺, Cu⁺⁺, B⁺⁺⁺ as well as CO₃⁻⁻, SO₄⁻⁻, PO₄⁻⁻⁻, NO₃⁻. Due to the fact that salts or acids and bases having the ions Na⁺, K⁺, Ca⁺⁺, Mg⁺⁺, Fe⁺⁺⁺, Zn⁺⁺, Cu⁺⁺, B⁺⁺⁺ as well as CO₃⁻⁻, SO₄⁻⁻, PO₄⁻⁻⁻, NO₃⁻ are contained in the liquid synthetic culture medium, it is ensured that any foreign ions possibly present in the fungi or bacteria in addition to carbon, hydrogen, nitrogen and sulphur will be safely provided in sufficient quantities so as to ensure high yields besides rapid growth.

In order to further improve the yield, a natural or synthetic carrier having a large internal surface area, in particular silicate, layered silicate, zeolite, bentonite, burnt clay, diatomaceous earth, synthetics or the like, is used as said inert carrier. By using an inert carrier having a large internal surface area, it is feasible to improve the yield in the method according to the invention by at least 50% as opposed to conventional methods, which are carried out without inert carriers having large internal surfaces areas. Such increases in yield not only render the method more economical, but also ensure that sufficient quantities of the desired end products of the isotopically labelled secondary metabolic products will be produced so as to enable the same to be used in a suitable manner as internal standards in analyses, or even in metabolic studies.

According to the invention, the greatest improvements in yield will, in particular, be obtained in that an aluminium silicate, e.g. diatomaceous earth, in particular kieselguhr, isolute HM-N or a zeolite, or a layered silicate, in particular a vermiculite, from the group of mica minerals is used in natural or treated form as said inert carrier. With these substances, the surface properties such as surface tension, porosity and the like are, in particular, suitable to achieve especially good turnover rates on the carrier surfaces. In an analogous manner, the use of inert synthetic carriers, which may be selected from foamed materials, polyamide, silicone, polyethylene, polypropylene, polytetrafluoroethylene, polyester or the like, will allow for accordingly large improvements in yield, whereby the use of natural carriers having large internal surface areas, or the use of synthetic carriers, will produce similarly enhanced yields as a function of the metabolic products to be produced.

For as rapid a method control as possible at a simultaneously high yield, the invention is further developed to the extent that the production is realized at temperatures ranging between 3 and 45° C., in particular between 10 and 35° C. In this context, it has partially turned out to be favourable that a production method is not always conducted at a constant temperature, but that temperature variations within the indicated limits may also lead to improved yields or accelerated reaction rates or elevated turnover numbers.

In order to obtain an end product as pure as possible, the method according to the invention is conducted in a manner that the isotopically labelled secondary metabolic products are recovered from the liquid synthetic culture medium by extraction and concentration, for instance by a combination of steps like solid/liquid-liquid/liquid extraction, centrifugation, filtration and evaporation. After the recovery of the isotopically labelled secondary metabolic products, it has turned out to be advantageous to subject these products to a further purification procedure, wherein, according to the invention, chromatographic methods and, in particular, column chromatography, preparative thin-layer chromatography, ion chromatography, affinity chromatography, exclusion chromatography and/or preparative high-pressure liquid chromatography are preferably used as purification procedures. Such a reprocessing method and purifying procedure will render feasible the recovery of isotopically labelled secondary metabolic products of fungi and bacteria, in which at least 95% of the carbon atoms, nitrogen atoms and/or sulphur atoms have been replaced with the respective stable isotopes, thus enabling the recovery of products with appropriate mass differences relative to their natural analytes so as to be sufficiently distinguishable from naturally occurring, heavy isotopes, for instance in a liquid chromatography with mass-spectrometric detection (LCMS), and, hence, for instance, allow for the provision of stable, clearly identifiable internal standards in such analyses.

The invention further aims to provide an isotopically labelled secondary metabolic product of fungi and bacteria, which comprises substantially all, in particular at least 95%, of the carbon atoms, nitrogen atoms and/or sulphur atoms replaced by stable isotopes.

According to a further development of the invention, such isotopically labelled secondary metabolic products of fungi or bacteria can be used as internal standards in analytics, for metabolic studies in animal feeding tests, for metabolic studies, for elucidating metabolic cycles, degradation paths and/or degradation periods as well as intercalations. For all of the mentioned purposes of use, it is of essential importance to have obtained a stable and clearly detectable standard, or a clearly detectable and trackable substance, in the test scheme or degradation scheme in order to be able to precisely reproduce the individual method or processing steps.

According to a further development, mycotoxins, in particular trichothecenes such as nivalenol, deoxynivalenol, 3-acetyl-deoxynivalenol, 15-acetyl deoxynivalenol, fusarenon-X, T-2 toxin, HT-2 toxin, DAS, fumonisins such as fumonisin B1, B2 or B3, ochratoxins such as ochratoxin A, B, C or D, zearalenones, moniliformin or aflatoxins such as aflatoxin B1, B2, G1 or G2 are used as metabolic products in analytical methods or in metabolic studies, degradation paths and the like. Mycotoxins are of increasing importance in the etiology of animal diseases, and it is necessary to technically produce sufficient quantities of such substances in order to be able to subsequently carry out the respective toxicological veterinary examinations by using chemical substances as distinct and pure as possible. Since mycotoxins constitute serious health risks to men and animals, their analytics is a theme of global interest, since, in particular, many countries have already developed guide and limit values for the tolerance of such substances. The detection and quantification of such mycotoxins via the use of internal standards that are precisely detectable and, hence, enable the quantitative analysis of the respective toxin constitute an important advance in the detection of such noxious substances.

Similarly, also the quantitative detection or tracking of toxins and, above all, as in correspondence with a further development of the invention, endoxins and exotoxins, in particular bacterial toxins of Escherichia coli sp., Salmonella sp., Clostridium sp., Bacillus sp. or Staphylococcus sp., is of vital interest for the public health and for the detection of harmful substances in food and semi-luxury food. Also the use of metabolic products such as antibiotics and, in particular, antibiotics formed of actinomycetes, like tetracyclines, streptomycines or aminoglycosides, antibiotics formed of Bazillus sp., like bacitracin or polymyxin, antibiotics formed of penicillium, like penicillin or griseofulvin, or cephalosporins formed of cephalosporium, is increasing in importance, in particular in the event of diseases or for the detection of such substances in food and semi-luxury food, wherein it also holds for these substances that substances rendering feasible the quantitative detection of metabolic products like antibiotics are of vial interest for the general public.

According to a further development, pure substances having labelling degrees of ¹³C, ¹⁵N, or ³³S or ³⁴S are used as metabolic products, thus, on the one hand, enabling the clear differentiation from unlabelled substances or metabolic products and, on the other hand, also safely ensuring the differentiation from naturally occurring isotopes and, finally, providing a substance to be precisely trackable in analyses and detection processes.

In the following, the invention will be explained in more detail by way of examples illustrating the production of highly isotopically labelled metabolic products.

### EXAMPLE 1

### Production of Highly Isotopically Labelled [U-¹³C₁₅]-deoxynivalenol (DON)

To produce completely labelled ¹³C-DON, a fusarium fungus, namely Fusarium graminearum, is inoculated on an inert carrier material, namely vermiculite, and incubated in a synthetic culture medium consisting of 0.5 g K₂HPO₄, 2.0 g NaNO₃, 0.7 g MgSO₄.7H₂O, 2.0 g KCl, 15 g D-[U-¹³C₆]glucose, 1.5 g NH₄H₂PO₄, 15 mg Fe(II) SO₄*7H₂O or 20 mg ZnSO₄*7H₂O and containing D-[U-¹³C₆]glucose as the sole carbon source. After 5 weeks at about 28° C., the toxin-containing material is extracted with ethyl acetate and subsequently purified to standard quality (purity >98%) by means of extraction, chromatography and crystallization.

About 40 ml incubation medium is prepared per production formulation. After this, pooled toxin-containing material is further processed. From 1000 ml formulation or batch, between 5 and 50 mg [7.5 to 17.5 mg] completely labelled [U-¹³C₁₅]-DON is obtained.

The purified product was characterized by the following analytical processes:

¹H NMR and ¹³C-NMR

LC-MS/MS Q Trap for determining the ¹³C isotope portion Determination of purity and concentration against reference materials using UV/VIS and HPLC-DAD

### Adjustment of Concentration

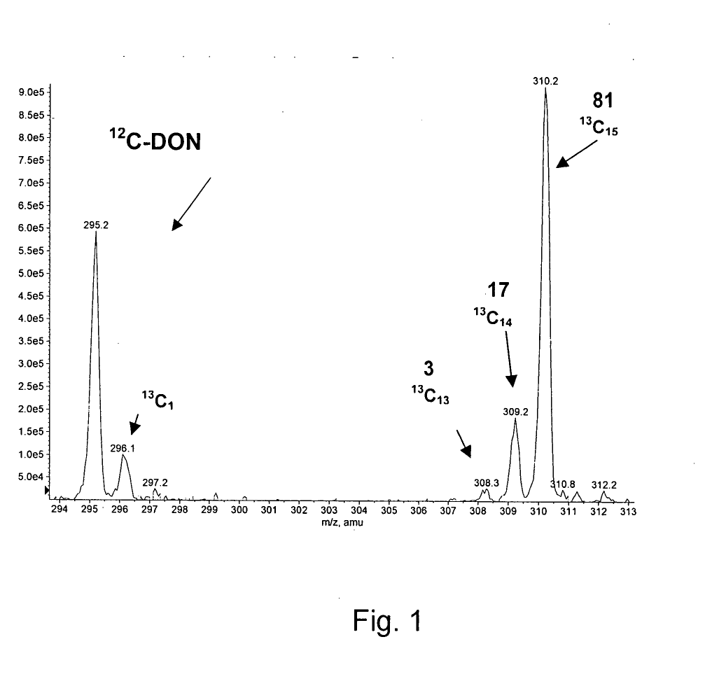

Quality control using UV/VIS; HPLC-DAD; LC-MS/MS A Trap Such a highly isotopically labelled ¹³C₁₅-deoxynivalenol (¹³C₁₅-DON) may, for instance, be used as an internal standard. Such an internal standard has a molecular mass that is heavier by exactly 15 g/mol, its signal in the mass spectrum (FIG. 1), thus, appearing exactly 15 amu higher than the signal of the analyte. Since all of the other chemical and physical properties of the ¹³C-labelled DON are identical with those of the analyte, such an internal standard shows exactly the same fragmentation as the analyte, also the ionization of the substance is identical and, hence, even the ionization yield. This means that the signal heights of the fragments or substance ions become absolutely comparable between the internal standard and the analyte, and, because the concentration of the internal standard is known in an analysis, direct conclusions can be drawn as to the concentration of an analyte, which is why such a highly labelled deoxynivalenol constitutes a near-ideal internal standard.

FIG. 1 shows C₁₂-DON and C₁₃-DON in a collective mass spectrum. In addition to the molecular peaks of ¹³C₁₅-DON and ¹²C-DON at 295.2 and 310.2, respectively, FIG. 1 also indicates the distribution of the compounds in which not all of the C-atoms have been labelled and, hence, do not consist of one isotope species (isotopomers). In the case of naturally occurring deoxynivalenol, this is ¹³C₁-DON, which corresponds to the natural distribution between C₁₂ and C₁₃.

### EXAMPLE 2

### Production of Highly Isotopically Labelled ¹³C-Fumonisin

To produce a fumonisin completely labelled with ¹³C; 1000 ml of a liquid medium consisting of 0.5 g KH₂PO₄, 0.5 g KNO₃, 0.7 g MgSO₄.7H₂O, 2.0 g KCl, 17.5 g D-[U-¹³C₆]glucose, 1.5 g NH₄H₂PO₄, 15 mg Fe(II) SO₄*7H₂O and 20 mg ZnSO₄*7H₂O, with D-[U-¹³C₆]glucose as the sole carbon source, applied on 1×1×1 cm foam cubes, are inoculated with Fusarium moniliforme and incubated in an incubator at 28° C. and 70% relative humidity. After 3 weeks, the toxin-containing material is extracted with a solvent mixture containing 1:1 acetonitril:H₂O and subsequently purified to standard quality (purity >98%) by means of extraction and chromatographic steps such as ion exchange chromatography, flash column chromatography with silica gel, thin-layer chromatography and preparative HPLC.

In this manner, 80-240 mg ¹³C-fumonisins are obtained per 1000 ml formulation (HPLC-FLD).

### EXAMPLE 3

### Production of Highly Isotopically Labelled [U-¹³C₁₇]-3-acetyl deoxynivalenol

To produce highly isotopically labelled [U-¹³C₁₇]-3-acetyl deoxynivalenol, 1000 ml of a synthetic liquid medium consisting of 0.5 g KH₂PO₄, 0.5 g KNO₃, 0.7 g MgSO₄*7H₂O, 2.0 g KCl, 17.5 g D-[U-¹³C₆]glucose, 1.5 g NH₄H₂PO₄, 15 mg Fe(II) SO₄*7H₂O and 20 mg ZnSO₄*7H₂O and containing completely isotopically labelled [U-¹³C₆]-glucose as the sole carbon source, applied on diatomaceous earth, namely isolute HM-N, are inoculated with Fusarium graminearum and incubated in an incubator at 28° C. for 9 days. After 9 days, the toxin-containing material is harvested, extracted with acetonitril/H₂O azeotrope and subsequently purified to standard quality (purity >98%) by means of extraction, chromatography, crystallization, Buchi-MPLC and recrystallization. From one batch, about 15-50 mg of a highly pure end product can be obtained. The purity check is preformed by LC-UV analysis using a C18-capillary column.

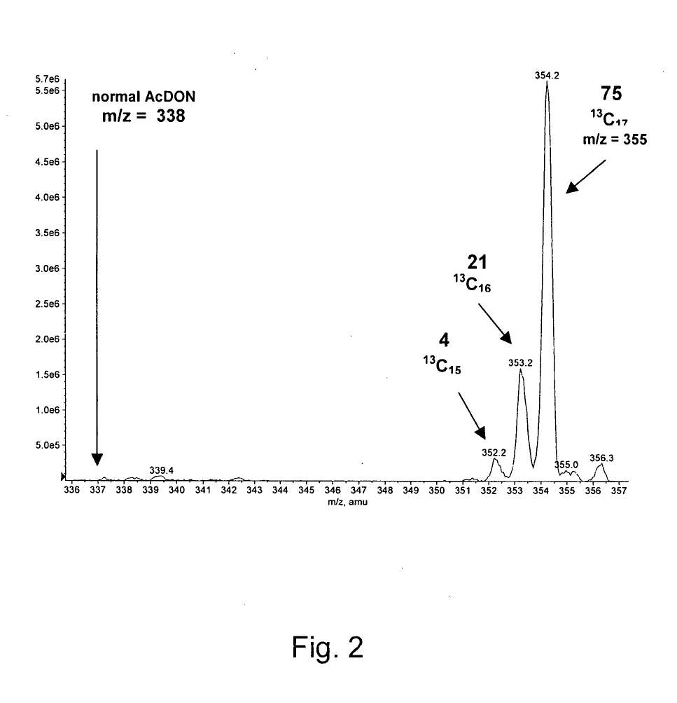

An isotopically labelled 3-acetyl deoxynivalenol produced in this manner has a molecular mass that is heavier by 17 g/mol than that of unlabelled or not thoroughly or incompletely labelled 3-acetyl deoxynivalenol. Incompletely labelled 3-acetyl deoxynivalenol has a molecular mass of M/z=338, whereas the completely isotopically labelled product has a molecular mass of 355. FIG. 2 shows the mass spectrum of pure ¹³C-3-acetyl-deoxynivalenol, from which it can be seen that the product has been labelled by 75% and the isotope distribution of the product is apparent. The distribution of the product and the incompletely labelled isotopomers in this case depends on the isotopic purity of the starting product, ¹³C₆-glucose, and can still be clearly shifted towards a completely labelled product when using completely pure ¹³C₆-glucose. From FIG. 2 it is, however, clearly apparent that isotopomers having less than 13 ¹³C-atoms are virtually absent such that ¹³C-3-acetyl-deoxynivalenol can also be perfectly used as an internal standard.

### EXAMPLE 4

### Production of Highly Isotopically Labelled [U-¹³C₁₇]-15-acetyl deoxynivalenol

To produce highly isotopically labelled [U-¹³C₁₇]-15-acetyl deoxynivalenol, a culture medium consisting of 0.5 g K₂HPO₄, 2.0 g NaNO₃, 0.7 g MgSO₄*7H₂O, 2.0 g KCl; 15 g D-[U-¹³C₆]glucose, 1.5 g NH₄H₂PO₄, 15 mg Fe(II) SO₄*7H₂O and 20 mg ZnSO₄*7H₂O is inoculated with Fusarium graminearum on a coarse-grained phyllosilicate carrier and incubated at 28° C. in an incubator. After 9 days, the toxin-containing material is harvested, extracted with ethyl acetate and subsequently purified to standard quality (purity >98%) by means of extraction, chromatography and crystallization. Alternatively to crystallization, a further purification step using preparative HPLC may also be applied.

About 30-60 mg highly pure target product can be obtained from one fermentation batch.

### EXAMPLE 5

### Production of Highly Isotopically Labelled [U-¹³C₁₅]-nivalenol or [U-¹³C₁₇]-fusarenon-X

To produce highly isotopically labelled [U-¹³C₁₅]-nivalenol or [U-¹³C₁₇]-fusarenon-X, a liquid medium consisting of 0.5 g K₂HPO₄, 2.0 g NaNO₃, 0.7 g MgSO₄*7H₂O, 2.0 g KCl, 15 g D-[U-¹³C₆]glucose, 1.5 g NH₄H₂PO₄, 15 mg Fe(II) SO₄*7H₂O or 20 mg ZnSO₄*7H₂O with [U-¹³C₆]-glucose as the sole carbon source and inert phyllosilicate is inoculated with Fusarium nivale and incubated at 28° C. for 5 weeks. After this, the toxin-containing material is extracted with methanol and methylene chloride and subsequently purified to standard quality (purity >98%) by means of extraction, chromatography and crystallization. Alternatively to crystallization, a further purification step using preparative HPLC may also be applied.

### EXAMPLE 6

### Production of Highly Isotopically Labelled [U-¹³C₂₀]-ochratoxin A

To obtain the target substance, the fungus Petromyces albertensis on an inert phyllosilicate carrier is fermented with a synthetic liquid medium consisting of 0.5 g K₂HPO₄, 2.0 g NaNO₃, 0.7 g MgSO₄*7H₂O, 2.0 g KCl, 15 g D-[U-¹³C₆]glucose, 1.5 g NH₄H₂PO₄, 15 mg Fe(II) SO₄*7H₂O or 20 mg ZnSO₄*7H₂O, which contains completely ¹³C-labelled glucose as the sole carbon source. The flasks are then incubated in an incubator for 6 weeks at 28° C. and 70% air moisture and subsequently extracted with toluene. The target substance is purified by column-chromatography and recrystallized as in the preceding Examples.

### EXAMPLE 7

### Production of Highly Isotopically Labelled [U-3C₁₈]-zearalenon

To produce highly isotopically labelled [U-¹³C₁₈]-zearalenon, 1000 ml of a liquid medium consisting of 0.5 g KH₂PO₄, 0.5 g KNO₃, 0.7 g MgSO₄*7H₂O, 2.0 g KCl, 17.5 g D-[U-¹³C₆]glucose, 1.5 g NH₄H₂PO₄, 15 mg Fe(II) SO₄*7H₂O and 20 mg ZnSO₄*7H₂O, with D-[U-¹³C₆]glucose as the sole carbon source, applied on porous burnt clay in granular form, namely Seramis or Lecca, is inoculated with Fusarium semitectrum and incubated in an incubator at 28° C. and 70% relative humidity. After 3 weeks, the toxin-containing material is extracted with pure petroleum ether and a petroleum ether/ethyl acetate mixture of 4:1 and 2:1 and subsequently purified to standard quality (purity >98) by means of extraction and chromatographic steps such as ion exchange chromatography, flash column chromatography with silica gel, thin-layer chromatography and preparative HPLC.

### EXAMPLE 8

### Production of Highly Isotopically Labelled ¹⁵N₅-rocquefortine C

To produce a rocquefortin C completely labelled with the nitrogen isotope ¹⁵N, 1000 ml of a liquid medium consisting of 0.8 g KH₂PO₄, 0.7 g MgSO₄*7H₂O, 1.0 g KCl, 17.5 g D-Glucose, 1.0 g ¹⁵NH₄¹⁵NO₃, 1.5 g NaH₂PO₄, 15 mg Fe(II) SO₄*7H₂O, 20 mg ZnSO₄*7H₂O, with ¹⁵NH₄¹⁵NO₃ as the sole nitrogen source, applied on coarse kieselguhr, are inoculated with Penicillium commune and incubated in an incubator at 12° C. and 70% air moisture. After 48 days, the toxin-containing material is extracted with an organic solvent consisting of 9:1 chloroform:methanol and subsequently purified to standard quality (purity >98%) by means of liquid-liquid extraction, flash column chromatography with silica gel, and preparative HPLC. Per 1000 ml formulation, 300 mg ¹⁵ N-rocquefortin C is obtained (HPLC-FLD).

### EXAMPLE 9

### Production of Highly Isotopically Labelled 15N₂-³³S-penicillin

To produce a penicillin completely labelled with the nitrogen isotope ¹⁵N and the sulphur isotope ³³S, 1000 ml of a liquid medium consisting of 1.0 g K₂HPO₄, 0.2 g MgCl₂, 20.0 g D-glucose, 1.0 g ¹⁵NH₄¹⁵NO₃, 0.5 g Na₂³³SO₄, 1.5 g Na₂HPO₄, 5 mg Fe(II) Cl₂, 5 mg ZnCl₂, with ¹⁵NH₄¹⁵NO₃ as the sole nitrogen source and Na₂³³SO₄ as the sole sulphur source, applied on small foam cubes, are inoculated with Penicillium notatum and incubated in an incubator at 28° C. and 70% air moisture. After 30 days, the toxin-containing material is extracted with ethyl acetate and subsequently purified to standard quality (purity >98%) by means of liquid-liquid extraction, flash column chromatography with silica gel, and preparative HPLC. Per 1000 ml formulation, 500 mg ¹⁵N₂-³³S-penicillin is obtained (HPLC-FLD).

1. A method for producing isotopically labelled secondary metabolic products of fungi or bacteria in a liquid synthetic culture medium, wherein the synthesis is carried out by immobilizing the fungi or bacteria on an inert carrier while adding a liquid synthetic culture medium in which substantially all of the carbon atoms, nitrogen atoms and/or sulphur atoms have been replaced by stable isotopes.

2. The method according to claim 1, wherein sugars or sugar alcohols, in particular D-[U-¹³C₆]-glucose, ¹³C-sucrose, ¹³C-gycerol and/or ¹³C-acetate are used as carbon sources in the liquid synthetic culture medium, ¹⁵N-amino acids, -nitrates, -ammonium compounds or -urea are used as nitrogen sources, ³³S- or ³⁴S-sulphates, -sulphides or -amino acids are used as sulphur sources.

3. The method according to claim 1, wherein the liquid synthetic culture medium additionally contains a mixture selected from inorganic salts or acids and bases having the ions Na⁺, K⁺, Ca⁺⁺, Mg⁺⁺, Fe⁺⁺⁺, Zn⁺⁺, Cu⁺⁺, B⁺⁺⁺ as well as CO₃⁻⁻, SO₄⁻⁻, PO₄⁻⁻⁻, NO₃⁻.

4. The method according to claim 1, wherein a natural or synthetic carrier having a large internal surface area, in particular silicate, layered silicate, zeolite, bentonite, burnt clay, diatomaceous earth, synthetics or the like, is used as said inert carrier.

5. The method according to claim 4, wherein an aluminium silicate, for instance a zeolite or a layered silicate, in particular a vermiculite, from the group of mica minerals is used in natural or treated form as said inert carrier.

6. The method according to claim 4, wherein foamed materials, polyamide, silicone, polyethylene, polypropylene, polytetrafluoroethylene, polyester or the like are used as said inert synthetic carrier.

7. The method according to claim 1, wherein the production is realized at temperatures ranging between 3 and 45° C., in particular between 10 and 35° C.

8. The method according to claim 1, wherein the isotopically labelled secondary metabolic products are recovered from the liquid synthetic culture medium by extraction and concentration, for instance by a combination of steps like solid/liquid-liquid/liquid extraction, centrifugation, filtration and evaporation.

9. The method according to claim 8, wherein chromatographic methods and, in particular, column chromatography, preparative thin-layer chromatography, ion chromatography, affinity chromatography, exclusion chromatography and/or preparative high-pressure liquid chromatography are used as purification processes.

10. The isotopically labelled secondary metabolic product of fungi and bacteria produced by a method according to claim 1, for the production of an internal standard in analytics, for metabolic studies in animal feeding tests, for metabolic studies, for clarifying metabolic cycles, degradation paths and/or degradation periods as well as intercalations.

11. The metabolic product according to claim 10, wherein mycotoxins, in particular trichothecenes such as nivalenol, deoxynivalenol, 3-acetyl-deoxynivalenol, 15-acetyl deoxynivalenol, fusarenon-X, T-2 toxin, HT-2 toxin, DAS, fumonisins such as fumonisin B1, B2 or B3, ochratoxins such as ochratoxin A, B, C or D, zearalenones, moniliformin or aflatoxins such as aflatoxin B1, B2, G1 or G2 are used as metabolic products.

12. The metabolic product according to claim 10, wherein toxins such as endoxins and exotoxins, in particular bacterial toxins of Escherichia coli sp., Salmonella sp., Clostridium sp., Bacillus sp. or Staphylococcus sp., are used as said metabolic product.

13. The metabolic product according to claim 10, wherein antibiotics and, in particular, antibiotics formed of actinomycetes, like tetracyclines, streptomycines or amino-glycosides, antibiotics formed of Bazillus sp., like bacitracin or polymyxin, antibiotics formed of penicillium, like penicillin or griseofulvin, or cephalosporins formed of cephalosporium, are used as said metabolic product.

14. The metabolic product according to claim 10 as a pure substance having a labelling degree with ¹³C, ¹⁵N, or ³³S or ³⁴S of at least 95%.
